(19) United States
(12) Patent Application Publication
(10) Pub. No.: US 2025/0017460 A1
(43) Pub. Date: Jan. 16, 2025

(54) IMAGE-GUIDED NAVIGATION SYSTEM FOR A VIDEO LARYNGOSCOPE

(71) Applicant: COVIDIEN LP, Mansfield, MA (US)

(72) Inventors: Mingxia Sun, Shanghai (CN); Qing Wang, Shanghai (CN)

(21) Appl. No.: 18/711,471

(22) PCT Filed: Dec. 10, 2021
(86) PCT No.: PCT/CN2021/137080

§ 371 Date: May 17, 2024

(51) Int. Cl. A61B 1/267; A61B 1/00

(52) U.S. Cl. CPC A61B 1/2673; A61B 1/00006; A61B 1/000094

(57) ABSTRACT

A video laryngoscope navigation system is provided that identifies an anatomical feature in an image signal from a camera of a video laryngoscope. Based on the identified anatomical feature, a steering indicator is overlaid on a displayed image of the image signal. In an embodiment, the steering indicator is representative of a steering direction for the camera to orient the camera towards the identified anatomical feature.

### CROSS-REFERENCE TO RELATED APPLICATION

The present application is a 371 national stage application that claims priority to PCT/CN2021/137080, which was filed on Dec. 10, 2021, the disclosure of which is hereby incorporated by reference in its entirety herein.

### BACKGROUND

The present disclosure relates generally to medical devices and, more particularly, to image-guided navigation using a video laryngoscope and related methods and systems.

In the course of treating a patient, a tube or other medical device may be used to control the flow of air, food, fluids, or other substances into the patient. For example, tracheal tubes may be used to control the flow of air or other gases through a patient's trachea and into the lungs, for example during mechanical ventilation. Such tracheal tubes may include endotracheal (ET) tubes, tracheostomy tubes, or transtracheal tubes. Laryngoscopes are in common use for the insertion of endotracheal tubes into the tracheas of patients during medical procedures. Laryngoscopes may include a light source to permit visualization of the patient's airway to facilitate intubation, and video laryngoscopes may also include an imager, such as a camera. A laryngoscope, when in use, extends only partially into the patient's airway, and the laryngoscope may function to push the patient's tongue aside to permit a clear view into the airway for insertion of the endotracheal tube.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In an embodiment, a video laryngoscope is provided. The video laryngoscope includes a handle and an arm and a camera coupled to the handle and producing an image signal. The video laryngoscope also includes a display that operates to display an image based on the image signal. The video laryngoscope also includes a processor that operates to receive the image signal; identify an anatomical feature in the image signal; determine a steering direction for the camera based on the identified anatomical feature; and overlay an indicator of the steering direction on the displayed image.

In an embodiment, a video laryngoscope navigation method is provided that includes the steps of receiving an image signal from a camera of a video laryngoscope; identifying an anatomical feature in the image signal; determining a steering direction for the camera based on the identified anatomical feature; and overlaying an indicator of the steering direction on the displayed image.

In an embodiment, a video laryngoscope navigation method is provided that includes the steps of receiving an image signal from a camera of a video laryngoscope; identifying at least one anatomical feature in the image signal; determining whether the identified at least one anatomical feature comprises a glottis or vocal cord; displaying a first type of steering indicator overlaid on the image when the identified at least one anatomical feature comprises a glottis or vocal cord; and determining a direction of the glottis or vocal cord when the identified at least one anatomical feature does not comprise a glottis or vocal cord and displaying a second type of steering indicator overlaid on the image based on the direction.

In an embodiment, a video laryngoscope navigation method is provided that includes the steps of receiving an image signal from a camera of a video laryngoscope; identifying a first anatomical feature in the image signal; determining a first steering direction of the camera towards a glottis or vocal cords based on the identified first anatomical feature; displaying an indicator overlaid on an image of the image signal based on the first steering direction; receiving an updated image signal; identifying a second anatomical feature in the updated image signal; determining a second steering direction of the camera towards a glottis or vocal cords based on the identified second anatomical feature; and displaying an updated indicator overlaid on an image of the updated image signal based on the second steering direction.

In an embodiment, an image-guided navigation system is provided. The system includes a display, a memory, and a processor that operates to receive a user input selecting a recorded video file from a video laryngoscope stored in the memory and activating a navigation review setting for the recorded video file; cause the recorded video file to be displayed on the display; while a frame of the recorded video file is being displayed on the display, identify an anatomical feature in the frame; determine a steering direction for a camera of the video laryngoscope based on the identified anatomical feature; and overlay an indicator of the steering direction on the displayed frame.

In an embodiment, an image-guided navigation method is provided that includes the steps of receiving a user input of a selected recorded video file from a video laryngoscope; activating a navigation review setting for the recorded video file; causing the recorded video file to be displayed on the display; while a frame of the recorded video file is being displayed on the display, identifying an anatomical feature in the frame; and determining a steering direction for a camera of the video laryngoscope based on the identified anatomical feature; and overlaying an indicator of the steering direction on the displayed frame.

Features in one aspect or embodiment may be applied as features in any other aspect or embodiment, in any appropriate combination. For example, features of a system, handle, controller, processor, scope, method, or component maybe implemented in one or more other system, handle, controller, processor, scope, method, or component.

### BRIEF DESCRIPTION OF THE DRAWINGS

Advantages of the disclosed techniques may become apparent upon reading the following detailed description and upon reference to the drawings in which:

FIG. 1 is a schematic illustration of a video laryngoscope that may be used in conjunction with the disclosed image-guided navigation system, according to an embodiment of the disclosure;

FIG. 2 is a flow diagram of a video laryngoscope navigation method, according to an embodiment of the disclosure;

FIG. 3 is a schematic illustration of relative positions of anatomical features of the upper airway;

FIG. 4 is a flow diagram of a video laryngoscope navigation method that includes different steering indicator types, according to an embodiment of the disclosure;

FIG. 5 is an example displayed image of a video laryngoscope with an overlaid steering indicator of a video laryngoscope navigation system, according to an embodiment of the disclosure;

FIG. 6 is an example displayed image of a video laryngoscope with an overlaid steering indicator of a video laryngoscope navigation system, according to an embodiment of the disclosure;

FIG. 7 is an example displayed image of a video laryngoscope with an overlaid steering indicator of a video laryngoscope navigation system, according to an embodiment of the disclosure;

FIG. 8 is a flow diagram of a video laryngoscope navigation method for determining steering direction based on identified anatomical features, according to an embodiment of the disclosure;

FIG. 9 is an example user interface to select a post-processing video laryngoscope navigation method, according to an embodiment of the disclosure;

FIG. 10 is a flow diagram of a post-processing video laryngoscope navigation method, according to an embodiment of the disclosure; and

FIG. 11 is a block diagram of a video laryngoscope system with image-guided navigation, according to an embodiment of the disclosure.

### DETAILED DESCRIPTION OF SPECIFIC EMBODIMENTS

A medical practitioner may use a laryngoscope to view a patient's upper airway to facilitate insertion of a tracheal tube (e.g., endotracheal tube, tracheostomy tube, or transtracheal tube) into the patient's trachea as part of an intubation procedure. Video laryngoscopes include a camera that is inserted into the patient's upper airway to obtain an image (e.g., still image and/or moving image, such as a video). The image is displayed during the intubation procedure to aid navigation of the tracheal tube through the vocal cords and into the trachea. Video laryngoscopes permit visualization of the vocal cords as well as the position of the endotracheal tube relative to the vocal cords during insertion to increase intubation success. In emergency situations, intubations may be required at any time of day with little time for preparation and, thus, can be performed by less experienced practitioners. Practitioner-related factors such as experience, device selection, and pharmacologic choices affect the odds of a successful intubation on the first attempt as well as a total time of the intubation procedure. Further, patient-related factors often make visualization of the airway and placement of the tracheal tube difficult.

Accordingly, the disclosed embodiments generally relate to a video laryngoscope that includes an image-guided navigation system to assist airway visualization, such as during insertion of an endotracheal tube. The image-guided navigation system uses images acquired by the camera of the video laryngoscope to identify one or more anatomical features in the acquired images and, based on the identified anatomical features, to generate steering indicators. In an embodiment, the steering indicator may be an arrow pointing towards a recommended steering direction of the video laryngoscope that is overlaid on the display of the live camera image. Using the steering indicator for navigation guidance, the operator can adjust the angle or grip of the video laryngoscope to reorient the laryngoscope camera towards particular anatomical features of the airway. In turn, the view of the reoriented laryngoscope camera permits improved visualization of the airway during intubation or other procedures. In one example, the steering indicators marks or flags the patient vocal cords and/or the opening between the vocal cords, i.e. the glottis. In an embodiment, the steering indicators can be used as navigation guidance for the insertion of airway devices.

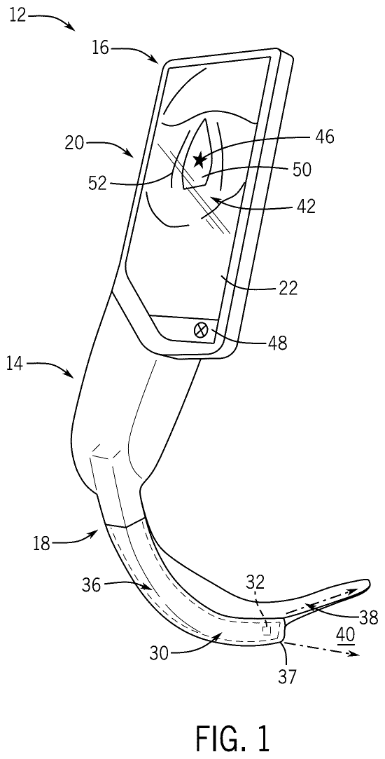

The present techniques relate to a video laryngoscope 12, as shown in FIG. 1, that includes an image-guided navigation system as in the disclosed embodiments. The video laryngoscope 12 includes an elongate handle 14, which may be ergonomically shaped to facilitate grip by a user. The video laryngoscope 12 extends from a proximal end 16 to a distal end 18 and also includes a display, e.g., a display assembly 20 having a display screen 22. As illustrated, the display assembly 20 is coupled to the proximal end 16 and extends laterally from the handle 14. In the illustrated embodiment, the display assembly 20 may be formed as an integrated piece with the handle 14, such that a housing of the display assembly 20 and an exterior of the handle 14 are formed from the same material. However, in other embodiments, the display assembly 20 may be formed as a separate piece and adhered or otherwise coupled, e.g., fixedly or pivotably, to the handle 14.

In an embodiment, the video laryngoscope 12 also includes a camera stick 30, which may be coupled to the handle 14 at the distal end 18 (cither fixedly or removably). In certain embodiments, the camera stick 30 may be formed as an elongate extension or arm (e.g., metal, polymeric) housing an image acquisition device (e.g., a camera 32) and a light source. The camera stick 30 may also house cables or electrical leads that couple the light source and the camera to electrical components in the body 14, such as the display 20, a computer, and a power source. The electrical cables provide power and drive signals to the camera 32 and light source and relay image signals back to processing components in the handle 14. In certain embodiments, these signals may be provided wirelessly in addition to or instead of being provided through electrical cables.

In use to intubate a patient, a removable and at least partially transparent blade 38 is slid over the camera stick 30 like a sleeve. The laryngoscope blade includes an internal channel or passage 36 sized to accommodate the camera stick 30 and to position the camera 32 at a suitable angle to visualize the airway. In the depicted arrangement, the passage 36 terminates at a closed end face 37 positioned such that a field of view 40 of the camera 32 is oriented through the closed end face 37. The laryngoscope blade 38 is at least partially transparent (such as transparent at the closed end face 32, or transparent along the entire blade 38) to permit the camera 32 of the camera stick 30 to acquire live images through the laryngoscope blade 38 and to generate an image signal of the acquired images. The camera 32 and light source of the camera stick 30 facilitate the visualization of an endotracheal tube or other instrument inserted into the airway.

In the illustrated embodiment, the display screen 22 displays an airway image 42, which may be a live image or, as discussed in certain embodiments, a recorded image. The image-guided navigation system of the video laryngoscope 12 uses the acquired airway image 42 to identify at least one anatomical feature in the image 24, determine a steering direction, and overlay an indicator 46 on the image 24 in real-time based on the steering direction. As shown, the display screen 22 may also activate an icon 48 that is displayed while the image-guided navigation is active.

The indicator 46 provides steering guidance to an operator of the video laryngoscope 12. In the illustrated embodiment, the image 42 shows the indicator 46 overlaid on a glottis 50 to mark a space or opening between vocal cords 52. Using the indicator 46, shown by way of example as a star, the operator can steer an endotracheal tube or other inserted device towards the indicator 46. Because intubations generally rely on manual manipulation and advancement of the endotracheal tube through the airway, the operator can use the indicator 46 as a target or, in other embodiments, as a direction guide for the manual advancement of the endotracheal tube as well as a guide for positioning the handle 14 of the video laryngoscope for better visualization. For example, if the indicator 46 is not positioned in a center of the image 42, the operator can adjust the handle 14 to center the indicator 46, thus centering the glottis 50 in the field of view 40.

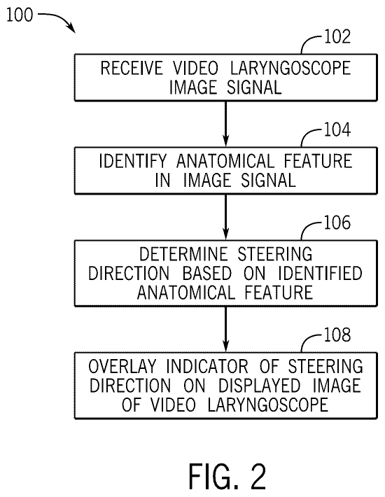

FIG. 2 is a flow diagram of a video laryngoscope navigation method 100 that can be used in conjunction with the video laryngoscope 12 and with reference to features discussed in FIG. 1, in accordance with an embodiment of the present disclosure. Certain steps of the method 100 may be performed by the video laryngoscope 12. The method 100 initiates with receiving an image signal that includes one or more images acquired by the camera 32 of the video laryngoscope 12 (block 102). One or more anatomical features are identified in the image signal (block 104), such as a mouth, tongue, epiglottis, supraglottis, vocal cord and glottis. The anatomical features may include passages and surrounding passage walls of the airway.

Based on the identified anatomical feature or features, the method 100 can determine a steering direction (block 106) and graphically render or generate an indicator of the steering direction (such as the indicator 46) that is overlaid on the image 42 that is displayed on the video laryngoscope (block 108). In an embodiment, the indicator of the steering direction can alert the operator that the video laryngoscope 12 is not positioned correctly to visualize a particular anatomical feature, such as the vocal cords. The operator can follow the guidance of the steering indicator to adjust a grip on the handle 14 of the video laryngoscope 12 to insert the camera 32 further into the airway and/or change an orientation of the camera 32. In an embodiment, the indicator of the steering direction can mark or highlight a particular anatomical feature.

As provided herein, the image-guided navigation system operates to identify one or more anatomical features in an image signal of the video laryngoscope 12. In an embodiment, the image-guided navigation system uses real-time video segmentation algorithm, machine learning, deep learning or other segmentation techniques, to identify anatomical features. In one example, the disclosed navigation system uses a video image segmentation approach to label or classify image pixels that are associated with anatomical features or not. One approach classifies the pixels that are associated with a moving object and subtracts a current image from a time-averaged background image to identify nonstationary objects. In another example, video segmentation represents image sequences through homogeneous regions (segments), where the same object carries the same unique label along successive frames. This is in contrast to techniques that independently compute segments for each frame, which are computationally less efficient. In another example, a superpixel segmentation algorithm uses real-time optical flow. Superpixels represent suggested object boundaries based on color, depth and motion. Each outputted superpixel has a 3D location and a motion vector, and thus allows for segmentation of objects by 3D position and by motion direction over successive images.

For example, the navigation system may use a machine learning model, such as a supervised or unsupervised model. In an embodiment, the feature identification model 54 may be built using a set of airway images and associated predefined labels for anatomical features of interest (which, in an embodiment, may be provided manually in a supervised machine learning approach). This training data, with the associated labels, can be used to train a machine classifier, so that it can later process the image signal.

Depending on the classification method used, the training set may either be cleaned, but otherwise raw data (unsupervised classification) or a set of features derived from cleaned, but otherwise raw data (supervised classification). In an embodiment, deep learning algorithms may be used for machine classification. Classification using deep learning algorithms may be referred to as unsupervised classification. With unsupervised classification, the statistical deep learning algorithms perform the classification task based on processing of the data directly, thereby eliminating the need for a feature generation step. Features can be extracted from the set using a deep learning convolutional neural network, and the images can be classified using logistic regression, random forests, SVMs with polynomial kernels, XGBoost, or a shallow neural network. A best-performing model that most accurately correctly labels anatomical features can be selected.

The disclosed techniques can be used to one, two, three, four, or more anatomical features in a same image or successive images acquired during a laryngoscope procedure. In an embodiment, the disclosed segmentation techniques and/or machine learning models can identify anatomical features that correspond to the various structures of the upper airway. An anatomical model defining position relationships between the various structures can be used as part of feature identification. For example, feature identification can include determining a position and direction of the vocal cords based on the relative position of anatomical features in oropharynx that can be captured in the video laryngoscope image signal.

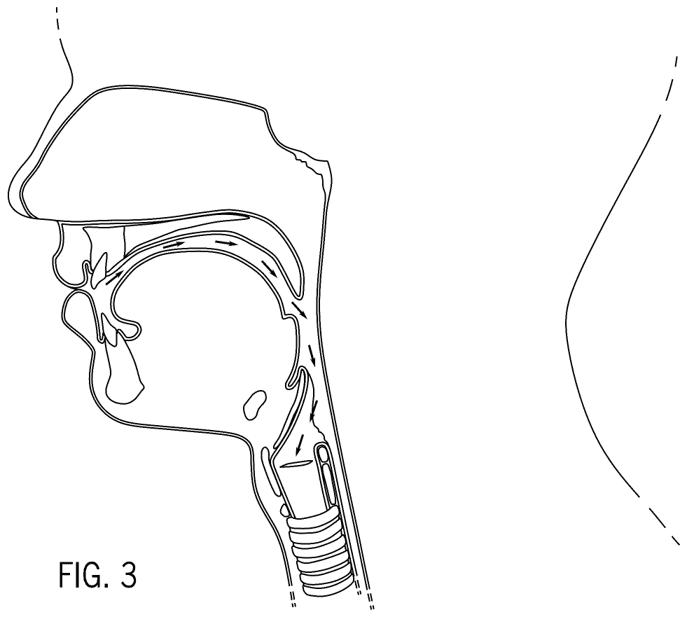

During insertion distally into the airway, shown by the path of the arrows in FIG. 3, anatomical features in the oral cavity and upper airway across the intubation path are encountered successively, such that the tongue is present in the image signal before images of the epiglottis, supraglottis, vocal cord and glottis are captured. Thus, the feature identification techniques can use relative positioning information to identify anatomical features. In one example, labeling of an identified feature can conform to predefined relative positioning such that features in an image signal cannot be labeled in a manner contrary to the anatomy, e.g., with a tongue positioned between the supraglottis and vocal cords.

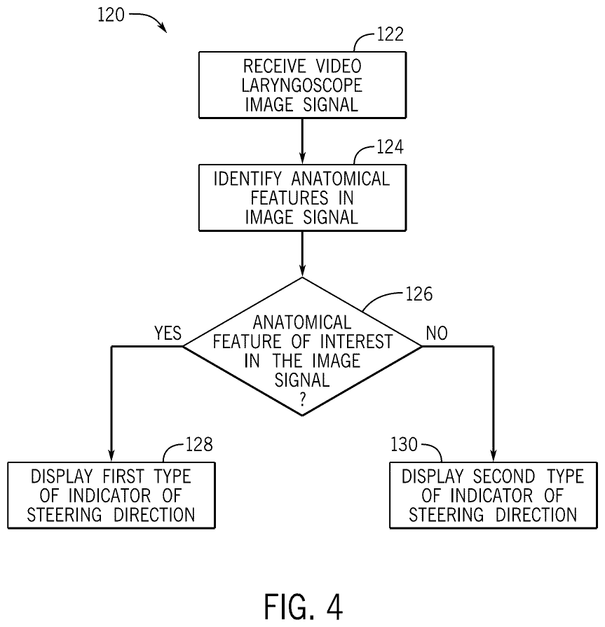

FIG. 4 is a flow diagram of a video laryngoscope navigation method 120 that can be used in conjunction with the video laryngoscope 12 and with reference to features discussed in FIGS. 1-2, in accordance with an embodiment of the present disclosure. Certain steps of the method 120 may be performed by a processor of the video laryngoscope 12. The method 120 initiates with receiving an image signal that includes one or more images acquired by the camera 32 of the video laryngoscope 12 (block 122). One or more anatomical features are identified in the image signal (block 124), such as a mouth, tongue, epiglottis, supraglottis, vocal cord and glottis. The feature identification can be performed as generally discussed with respect to FIGS. 2-3.

In certain embodiments, the video laryngoscope navigation method 120 facilitates operator-directed manipulation of the laryngoscope handle 14 to reorient and/or reposition the camera 32 to view the vocal cords to permit insertion of a medical device through the glottis distally into the tracheal passage. Thus, the method 120 can determine if a particular anatomical feature of interest, such as the vocal cords and/or glottis, is present in the image (block 126).

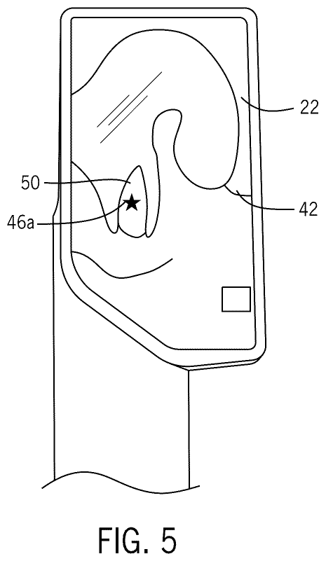

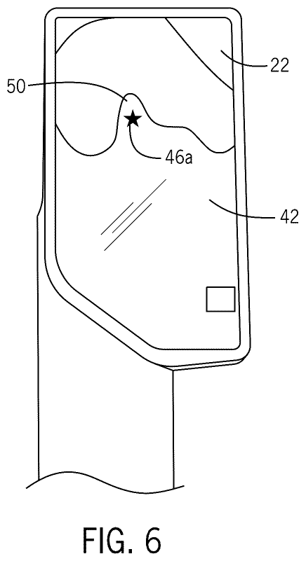

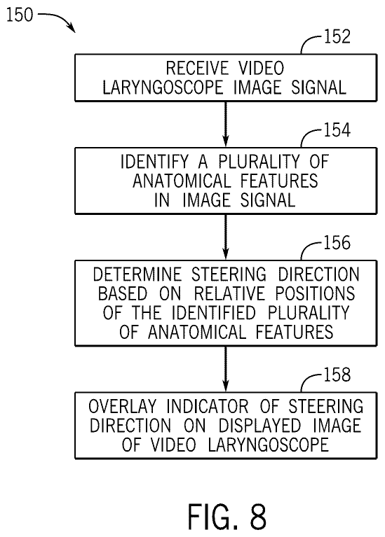

If the anatomical feature of interest is identified in the image signal, the method 120 activates display a first type of indicator of the steering direction (block 128), such as a star, bullseye, highlighted circle, or other indicator 46 that can be overlaid on or around the identified anatomical feature of interest (see FIGS. 5-6). The indicator 46 may act as a steering target for manipulation of an endotracheal tube into the camera field of view and towards the indicator 46. Automatic identification and labeling of a particular anatomical feature of interest can increase intubation efficiency and speed. The appearance of the indicator 46 provides information to the operator that the video laryngoscope 12 is properly positioned within the upper airway to visualize the vocal cords. In contrast, if the video laryngoscope 12 is not yet properly positioned, the method 120 activates display of a second, different, type of indicator 46 of the steering direction (block 130). For example, the second type of indicator 46 of the steering direction can be an arrow that points in an estimated direction of the vocal cords or a text-based message providing steering instructions. The estimated direction of the vocal cords can be based on patient anatomy models and relative positioning between different anatomical features as discussed with respect to FIG. 2 and FIG. 8.

The method 120 can iterate back to the start when updated images are received. Accordingly, the method can initially show an arrow-type indicator 46 as the video laryngoscope 12 is being inserted through the mouth and into the upper airway and while the vocal cords are not yet in the field of view 40. As soon as the vocal cords are identified in the image, the displayed indicator 46 switches from the second type to the first type. For example, the arrow stops displaying, and a star or other mark is overlaid on the identified vocal cords and/or glottis.

The disclosed image-guided navigation provides a user-friendly interface to assist inexperienced intubators in training who may not be familiar with the anatomical variations of vocal cords between patients and may not be able to quickly identify the vocal cords in laryngoscope image. However, an operator familiar with the user interface, and a particular indicator type marking the vocal cords and/or glottis, can quickly spot the indicator to guide insertion of an endotracheal tube. By way of example FIGS. 5-7 are example airway images showing detected objects with overlaid steering indicators 46.

FIG. 5 shows an example display screen 22 displaying the image 42 according to the disclosed embodiments in which the glottis 50 is labeled with the indicator 46a, e.g., a first type of indicator as discussed in FIG. 4, shown as a star. The indicator 46a can be scaled to fit entirely within, or around, the identified anatomical feature. For example, the indicator 46a can be scaled to cover less than 50% of the surface area of the glottis 50. In this manner, the glottis 50 is not obscured by the indicator 46a. In another embodiment, the indicator 46a may be rendered as an outlined shape, or may be rendered as a partially transparent shape through the glottis 50 or other anatomical feature can be viewed.

FIG. 6 is another example of a display screen 22 that shows a displayed image 42 of an airway from a different patient in which the vocal cords and glottis 50 have a different shape. The glottis 50 is identified with the indicator 46a to provide a steering direction for the operator for insertion of a device. The laryngoscope operator may not be familiar with a glottis anatomy as shown in FIG. 6, However, because the glottis 50 is automatically identified and labeled, even a less experienced operator can nonetheless quickly and efficiently manipulate an endotracheal tube towards and through the glottis 50 by aiming for the indicator 46a. It should be understood that the glottis is shown by way of example, and other or additional anatomical features may be marked using the star or similar indicator 46a to indicate a point to aim for in the image 42.

In an embodiment, the indicator 46a is activated upon the identification of the anatomical feature of interest, regardless of positioning of the anatomical feature within the image 42. That is, the anatomical feature of interest need not be in the center or center region of the image 42.

Depending on the position and depth of the laryngoscope insertion, only a subset of the anatomical features of the upper airway may be in the field of view 40 of the camera 32, and thus in the image signal, at one time. For example, a shallow insertion of the laryngoscope, only the tongue and portion of the airway above the epiglottis may be visible in the capture image. In deeper insertions, the epiglottis, supraglottis, or vocal cords may be captured by the camera image. However, even when the vocal cords are not in the captured image, the direction and position of the vocal cords can be determined from the set of anatomical features that are identified and the relative position of adjacent anatomical structures across the intubation path. The direction of glottis or handle movement towards the vocal cords will be displayed on the screen using the indicator 46, e.g., the indicator 46b.

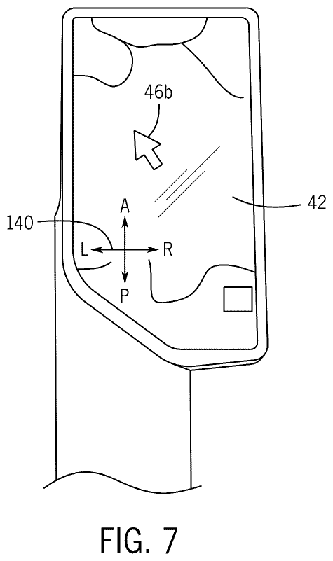

In another example, FIG. 7 shows an example display screen 22 displaying an image 42 in which there is no identified glottis or vocal cords. Thus, display of the indicator 46b, e.g., a second type of indicator as discussed in FIG. 4, is activated. The indicator 46b is shown as an arrow that originates in the center or center region of the image 42 and that points towards the vocal cords, which are out of frame and not present in the image 42. The arrow direction or steering direction towards the vocal cords or other anatomical feature of interest is determined using feature identification as discussed herein. To provide direction context, the display screen 22 may also include an icon 140 indicator left-right and anterior-posterior directions for the patient in the frame of reference of the image 42.

As discussed herein, the navigation system uses the laryngoscope image signal to generate a steering indicator 46 that can mark a particular anatomical feature of interest and/or provide instructions to reorient the laryngoscope handle 14 in an estimated direction towards the anatomical feature of interest. FIG. 8 is a flow diagram of a video laryngoscope navigation method 150 to determine a steering direction, e.g., towards a particular feature, using a set of already-visualized and identified anatomical features. The method 150 can be used in conjunction with the video laryngoscope 12 and with reference to features discussed in FIGS. 1-7, in accordance with an embodiment of the present disclosure. Certain steps of the method 120 may be performed by the video laryngoscope 12.

The method 150 initiates with receiving an image signal that includes one or more images acquired by the camera 32 of the video laryngoscope 12 (block 122). A plurality of anatomical features are identified in the image signal (block 154), such as a mouth, tongue, epiglottis, and/or supraglottis. Based on the identified anatomical features, the method 100 can determine a steering direction (block 156) and graphically render or generate the indicator 46 of the steering direction 46 that is overlaid on the image 42 that is displayed on the video laryngoscope (block 156). As new images are acquired, the method 150 can iterate back to block 152, and new steering indicators 46 can be generated that reflect a repositioned handle 14 and an updated direction for steering. Thus, when the steering indicator 46 is an arrow, the angle of the arrow can change as the operator repositions the handle 14. When the anatomical feature of interest enters the field of view 40 and is identified, the arrow can be deactivated, and the star, bullseye, or other steering indicator 46 marking the anatomical feature of interest can be activated.

In an embodiment, the disclosed techniques may incorporate an anatomy model with the identifiable anatomical features. Because different patients have different passage sizes, structure sizes, and different relative positioning, the anatomy model can estimate passage size and feature size based on the image signal and extrapolate positions and sizes of other features not in the image signal using population data. For example, patients having an upper airway passage within a particular estimated diameter range may typically exhibit a particular distance range between the epiglottis and the vocal cords. Further, the airway curve may also be within a particular angle range. Thus, once a more proximal anatomical feature or features are visualized, the navigation system can estimate a steering direction towards the vocal cords based on the position of the visualized features in the image signal and the anatomy model. The direction of the arrow can be determined using the parameters (e.g., size and relative position estimates) determined based on the image signal that are provided to the anatomy model during the laryngoscope insertion. The steering direction can be mapped to the live image to provide a direction relative to displayed anatomical features in the live image, which can be the laryngoscope operator's frame of reference. Thus, the operator can move the handle 14 in the direction of the arrow to move the camera 32 towards the vocal cords.

While certain embodiments of the disclosure are discussed in the context of live or real-time image-guided navigation, the disclosed embodiments may also be used for post-processing of recorded images acquired during laryngoscope procedures. The disclosed image-guided navigation techniques may not be available on a particular laryngoscope, or the image-guided navigation can be deactivated. Certain laryngoscope operators may prefer not to have the field of view 40 of the camera 32 obscured by steering indicators. However, recorded images acquired from a laryngoscope procedure can be assessed in a navigation playback mode. The disclosed playback embodiment may be used for training or to evaluate laryngoscope procedures retrospectively. For example, the path of the inserted endotracheal tube in the field of view 40 of the camera 32 can be viewed relative to a glottis that is marked with a steering indicator 46.

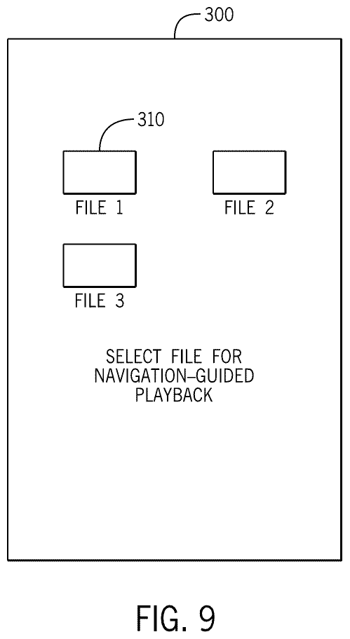

FIG. 9 shows an example user interface 300 for selecting recorded image files 310 for post-processing in a navigation playback mode to activate the image-guided navigation and in which steering indicators are overlaid on displayed frames of the recorded image files. The user interface 300 may be accessible from the video laryngoscope or part of a separate device that include features of the image-guided navigation system as discussed herein. In an embodiment, the recorded image files 310 are files that have been acquired and recorded without any image-guided navigation. However, the recorded image files 310 can be post-processed using image-guided navigation as provided herein. That is, the anatomical features are identified in the recorded image files 310, and the steering indicators 46 are overlaid on the frames of the recorded image files 310.

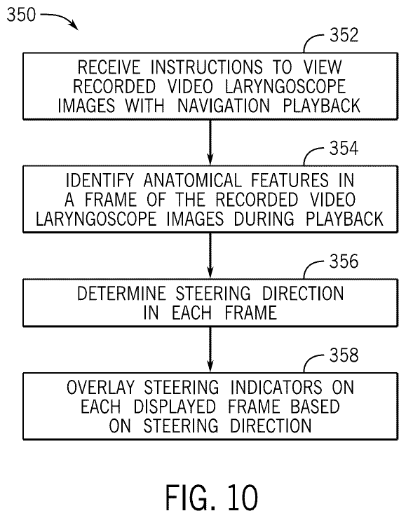

FIG. 10 is a flow diagram of a video laryngoscope playback navigation method 300 used for post-processing of recorded laryngoscope procedures. The method 350 can be used in conjunction with the video laryngoscope 12 or a separate device and with reference to features discussed in FIGS. 1-9. In the navigation playback mode, after a particular recorded video laryngoscope image file is selected (block 352), anatomical features are identified in successive frames of the recorded image file according to the disclosed image-guided navigation techniques (block 354). Using each successive frame as input, a steering direction is determined (block 356), and the steering indicators are overlaid on the respective frames as they are displayed (block 308). In an embodiment, the identified anatomical features can also be flagged in the displayed frames. The recorded image files 310 can be re-recorded with the overlaid steering indicators 46 for training purposes.

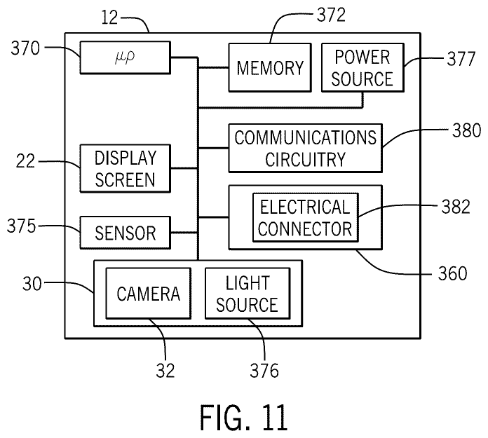

FIG. 11 illustrates a block diagram of the video laryngoscope 12. The block diagram illustrates control circuitry and hardware carried in the video laryngoscope 12, including a processor 370, a hardware memory 372, the laryngoscope camera 32 and a laryngoscope light source 376. The processor 370 may execute instructions stored in the memory 372 to send to and receive signals from the camera 32 and to illuminate the light source 376. The received camera signals include video signals (e.g., still images at a sufficiently rapid frame rate to create a video) that are processed and displayed on the display screen 22 of the display assembly 20 (see FIG. 1). The user may provide inputs via a sensor 75 (e.g., a capacitive touch screen sensor on the display screen 22, or mechanical or capacitive buttons or keys on the handle 14) to provide user inputs that are provided to the processor 70 to control settings or display characteristics. In certain embodiments, additional user input devices are provided, including one or more switches, toggles, or soft keys.

The video laryngoscope 12 may also include a power source 377 (e.g., an integral or removable battery) that provides power to one or more components of the laryngoscope 12. The video laryngoscope 12 may also include communications circuitry 380 to facilitate wired or wireless communication with other devices. In one embodiment, the communications circuitry may include a transceiver that facilitates handshake communications with remote medical devices or full-screen monitors. The communications circuitry 380 may provide the received images to additional monitors in real time.

The processor 370 may include one or more application specific integrated circuits (ASICs), one or more general purpose processors, one or more controllers, one or more programmable circuits, or any combination thereof. For example, the processor 70 may also include or refer to control circuitry for the display screen 22 or the laryngoscope camera 32. The memory 372 may include volatile memory, such as random access memory (RAM), and/or non-volatile memory, such as read-only memory (ROM). In one embodiment, the received signal from the laryngoscope camera 32, e.g., image data comprising one or more images, may be processed, to provide image-guided navigation according to stored instructions executed by the processor 370. Further, the image may be displayed with overlaid indicators or markings. The image data may be stored in the memory 372, and/or may be directly provided to the processor 370. Further, the image data for each patient intubation may be stored and collected for later review. The memory 372 may include stored instructions, code, logic, and/or algorithms that may be read and executed by the processor 370 to perform the techniques disclosed herein.

While the present techniques are discussed in the context of endotracheal intubation, it should be understood that the disclosed techniques may also be useful in other types of airway management or clinical procedures. For example, the disclosed techniques may be used in conjunction with placement of other devices within the airway, secretion removal from an airway, arthroscopic surgery, bronchial visualization past the vocal cords (bronchoscopy), tube exchange, lung biopsy, nasal or nasotracheal intubation, etc. In certain embodiments, the disclosed visualization instruments may be used for visualization of anatomy (such as the pharynx, larynx, trachea, bronchial tubes, stomach, esophagus, upper and lower airway, ear-nose-throat, vocal cords), or biopsy of tumors, masses or tissues. The disclosed visualization instruments may also be used for or in conjunction with suctioning, drug delivery, ablation, or other treatments of visualized tissue and may also be used in conjunction with endoscopes, bougies, introducers, scopes, or probes. Further, the disclosed techniques may also be applied to navigation and/or patient visualization using other clinical techniques and/or instruments, such as patient catheterization techniques. By way of example, contemplated techniques include cystoscopy, cardiac catheterization, catheter ablation, catheter drug delivery, or catheter-based minimally invasive surgery.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A video laryngoscope navigation method, comprising:
receiving an image signal from a camera of a video laryngoscope;
identifying an anatomical feature in the image signal;
determining a steering direction for the camera based on the identified anatomical feature; and
overlaying an indicator of the steering direction on the displayed image.

2. The method of claim 1, wherein the identified anatomical feature comprises a glottis and/or vocal cords.

3. The method of claim 1, wherein the steering direction is towards a glottis and/or vocal cords.

4. The method of claim 1, wherein the indicator of the steering direction is at least partially overlaid on the identified anatomical feature.

5. The method of claim 1, further comprising:
determining that a glottis and/or vocal cords is not identified in the image signal; and
displaying the indicator of the steering direction as an arrow pointing towards an estimated direction of the glottis and/or vocal cords.

6. The method of claim 1, further comprising:
determining that a glottis and/or vocal cords are the identified anatomical feature in the image signal; and
displaying the indicator of the steering direction positioned on the glottis and/or vocal cords or to highlight the glottis and/or vocal cords.

7. The method of claim 1, comprising identifying the anatomical feature based on image segmentation of the image signal.

8. The method of claim 1, wherein the image signal is a live image signal.

9. The method of claim 1, wherein the image signal is a recorded image signal.

10. The method of claim 1, comprising overlaying the indicator of the steering direction on the displayed image such that the identified anatomical feature is not covered by the indicator.

11. A video laryngoscope navigation method, comprising:
receiving an image signal from a camera of a video laryngoscope;
identifying at least one anatomical feature in the image signal;
determining whether the identified at least one anatomical feature comprises a glottis or vocal cord;
displaying a first type of steering indicator overlaid on the image when the identified at least one anatomical feature comprises a glottis or vocal cord; and
determining a direction of the glottis or vocal cord when the identified at least one anatomical feature does not comprise a glottis or vocal cord and displaying a second type of steering indicator overlaid on the image based on the direction.

12. The method of claim 11, wherein the first type of steering indicator is overlaid on the at least one identified anatomical feature.

13. The method of claim 11, wherein the second type of steering indicator is an arrow with an end at a center of the image and pointing in the direction of the glottis or vocal cord.

14. The method of claim 11, wherein the second type of steering indicator is a text indicator.

15. The method of claim 11, wherein the at least one identified anatomical feature comprises a plurality of anatomical features and wherein the direction of the glottis or vocal cord is determined based on relative positions of the plurality of anatomical features to one another.

16. A video laryngoscope navigation method, comprising:
receiving an image signal from a camera of a video laryngoscope;
identifying a first anatomical feature in the image signal;
determining a first steering direction of the camera based on the identified first anatomical feature;
displaying an indicator overlaid on an image of the image signal based on the first steering direction;
receiving an updated image signal;
identifying a second anatomical feature in the updated image signal;
determining a second steering direction of the camera based on the identified second anatomical feature; and
displaying an updated indicator overlaid on an image of the updated image signal based on the second steering direction.

17. The method of claim 16, wherein the indicator is an arrow pointing towards a glottis or vocal cords.

18. The method of claim 16, wherein identifying the first anatomical feature comprises identifying a plurality of anatomical features in the image signal and identifying the first anatomical feature based on relative positions of the identified plurality of anatomical features.

19. The method of claim 16, wherein the identified first anatomical feature is a mouth or tongue and the identified second anatomical feature is structure of the larynx.

20. An image-guided navigation method, comprising:
receiving a user input of a selected recorded video file from a video laryngoscope;
activating a navigation review setting for the recorded video file;
causing the recorded video file to be displayed on the display;
while a frame of the recorded video file is being displayed on the display, identifying an anatomical feature in the frame; and
determining a steering direction for a camera of the video laryngoscope based on the identified anatomical feature; and
overlaying an indicator of the steering direction on the displayed frame.

21. The method of claim 20, wherein the identified anatomical feature comprises a glottis and/or vocal cords.

22. The method of claim 21, wherein the indicator of the steering direction is positioned on the glottis and/or vocal cords.

23. The method of claim 20, wherein the indicator of the steering direction comprises an arrow pointing towards the glottis and/or vocal cords.

24. The method of claim 20, wherein the recorded video file is stored in the memory without any overlaid indicator of the steering direction.

25. The method of claim 20, the recorded video file is stored in the memory of the video laryngoscope.
